(19) United States
(12) Patent Application Publication
(10) Pub. No.: US 2003/0149272 A1
(43) Pub. Date: Aug. 7, 2003

(54) Method for preparing a polyaromatic compound

(76) Inventors: Henri-Jean Cristau, Saint Aunes (FR); Corinne Bouchitte, Le Pontet (FR); Marc Taillefer, Vailhauques (FR); Jean-Francis Spindler, Lyon (FR); Thierry Schlama, Dardilly (FR)

Correspondence Address: Kevin McVeigh, Intellectual Property Department, Rhodia Inc CN 7500, 259 Prospect Plains Road, Cranbury, NJ, 08512-7500, US

(21) Appl. No.: 10/203,111

(22) PCT Filed: Feb. 14, 2001
(86) PCT No.: PCT/FR01/00424

(30) Foreign Application Priority Data
Feb. 14, 2000 (FR) 00/01787

(51) Int. Cl. C07D 333/28; C07C 17/26; C07C 17/38; C07D 213/26; C07D 307/02

(52) U.S. Cl. 546/346; 570/237; 549/81; 549/504

(57) ABSTRACT

The invention concerns a method for preparing a polyaromatic compound comprising at least a chain formation of two aromatic cycles. The method for preparing a polycyclic aromatic compound comprising at least a chain formation of two aromatic cycles is characterised in that it consists in reacting an aromatic compound bearing a leaving group and an alkaline organometallic compound, in the presence of an efficient amount of a nickel catalyst, said element being optionally complexed with at least a co-ordination agent or ligand.

The subject-matter of the present invention is a preparation process for a polyaromatic compound.

The invention relates in particular to a compound of biphenyl type.

In the following description of the present invention, by “polycyclic aromatic compound” is meant a compound comprising at least a linkage of two carbocyclic and/or heterocyclic aromatic cycles.

By “aromatic compound” is meant the standard notion of aromaticity as defined in the literature, in particular by Jerry MARCH, Advanced Organic Chemistry, 4^{th} edition, John Wiley and Sons, 1992, pp. 40 et seq.

In a simplified manner, the expression “aryl” will describe, and “Ar” will symbolize, all aromatic compounds whether they be carbocyclic aromatic compounds or heterocyclic aromatic compounds.

Structures of biaryl type are found in numerous molecules used in the field of agrochemicals in particular in herbicides, pesticides or in the field of pharmaceuticals. In particular, non-symmetrical biaryls (Ar-Ar′) constitute an important class of organic compounds possessing a biological activity.

Present in numerous natural molecules, structures of biaryl type are thus much-sought targets during the elaboration of total synthesis.

Moreover, they assume a greater interest in relation to the development of new organic materials such as organic semiconductors or liquid crystals which possess polyfunctional units of biaryl type.

The synthesis of non-symmetrical biaryls is more complex than that of symmetrical biaryls.

A standard approach is to effect the coupling of an aryl halide or an aryl sulphonate and an organometallic aryl derivative, the reaction being catalysed by a palladium catalyst [S. P. Stanforth, Tetrahedron 54, pp. 263-303 (1998)].

The aim of the present invention is to provide another economically attractive process permitting in particular access to asymmetrical biaryls.

There has now been found, and it is this that constitutes the subject-matter of the present invention, a preparation process for a polycyclic aromatic compound comprising at least two linked aromatic cycles characterized in that it involves reacting an aromatic compound bearing a parting group and an alkaline organometallic compound, in the presence of an effective quantity of a nickel catalyst, the said element being optionally complexed with at least one coordination agent or ligand.

According to the process of the invention, it was found that the use of this process was attractive as it involves the use of a catalyst based on nickel, which is relatively inexpensive compared with the palladium-based catalysts that are customarily used.

By virtue of the choice of catalyst according to the invention, it is possible to use, as halogenoaromatic compounds, a chloroaromatic compound which is a more accessible and less expensive compound than a bromoaromatic compound.

More precisely, the aromatic compound bearing at least one parting group, hereafter called “halogenoaromatic compound”, conforms to the general formula (I):

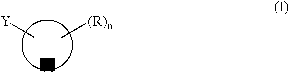

in which:

A symbolizes the remainder of a cycle forming all or part of an aromatic, monocyclic or polycyclic carbocyclic or heterocyclic system,

R, identical or different, represent substituents on the cycle,

Y represents a parting group, preferably a halogen atom or a sulphonic ester group of formula —OSO₂—R, in which R is a hydrocarbon group,

n represents the number of substituents on the cycle.

In the formula of the sulphonic ester group, R is a hydrocarbon group of any kind. However, given that Y is a parting group, it is attractive from an economic point of view for R to be simple in nature, and represent more particularly a linear or branched alkyl group having from 1 to 4 carbon atoms, preferably a methyl or ethyl group but it can also represent for example a phenyl or tolyl group or a trifluoromethyl group. Among the Y groups, the preferred group is a triflate group, which corresponds to a R group representing a trifluoromethyl group.

As preferred parting groups, a bromine or chlorine atom is preferably chosen.

The invention applies in particular to halogenoaromatic compounds conforming to the formula (I) in which A is the remainder of a cyclic compound, preferably having at least 4 atoms in the cycle, preferably 5 or 6, optionally substituted, and representing at least one of the following cycles:

a monocyclic or polycyclic aromatic carbocycle,

a monocyclic or polycyclic aromatic heterocycle comprising at least one of the heteroatoms O, N and S.

It will be specified, without thereby limiting the scope of the invention, that the optionally substituted remainder A represents the remainder:

1°—of a monocyclic or polycyclic aromatic carbocyclic compound.

By “polycyclic carbocylic compound” is meant:

a compound constituted by at least 2 aromatic carbocycles and forming between them ortho- or ortho- and pericondensed systems,

a compound constituted by at least 2 carbocycles only one of which is aromatic and forming between them ortho- or ortho- and pericondensed systems,

2°—of a monocyclic or polycyclic aromatic heterocyclic compound.

By “polycyclic heterocylic compound” is defined:

a compound constituted by at least 2 heterocycles containing at least one heteroatom in each cycle in which at least of the two cycles is aromatic and forming between them ortho- or ortho- and pericondensed systems,

a compound constituted by at one carbocycle and at least one heterocycle in which at least one of the cycles is aromatic and forming between them ortho- or ortho- and pericondensed systems.

More particularly, the optionally substituted remainder A represents one of the following cycles:

an aromatic carbocycle:

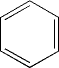

an aromatic bicycle comprising two aromatic carbocycles:

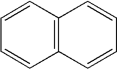

a partially aromatic bicycle comprising two carbocycles, one of which is aromatic:

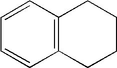

an aromatic heterocycle:

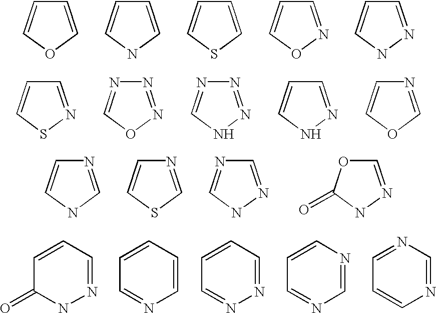

an aromatic bicycle comprising an aromatic carbocycle and an aromatic heterocycle.

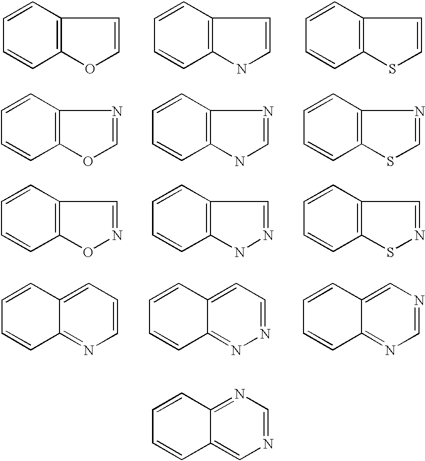

a partially aromatic bicycle comprising an aromatic carbocycle and a heterocycle:

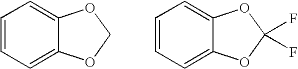

an aromatic bicycle comprising two aromatic heterocycles:

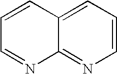

a partially aromatic bicycle comprising a carbocycle and an aromatic heterocycle:

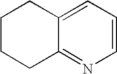

a tricycle comprising at least one carbocycle or an aromatic heterocycle:

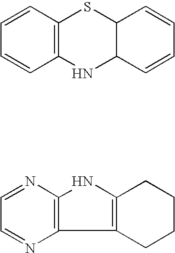

In the process of the invention, a halogenaromatic compound of formula (I) is preferentially used in which A represents an aromatic ring, preferably a benzene or napthalene ring.

The aromatic compound of formula (A) can bear one or more than one substituent.

The number of substituents present on the cycle depends on the carbon condensation of the cycle and on the presence or-not of unsaturated sites on the cycle.

The maximum number of substituents able to be borne by a cycle is easily determined by a person skilled in the art.

In the present text, “more than one” is generally taken to mean less than 4 substituents on an aromatic ring.

Examples of substituents are given below but this list is not limitative in character.

The R₁ group or groups, which may be identical or different, preferentially represent one of the following groups:

a linear or branched alkyl group, having from 1 to 6 carbon atoms, preferably from 1 to 4 carbon atoms, such as methyl, ethyl, propyl, isopropyl, butyl, isobutyl, sec-butyl, tert-butyl,

a linear or branched alkenyl or alkynyl group, having from 2 to 6 carbon atoms, preferably from 2 to 4 carbon atoms, such as vinyl, allyl,

a linear or branched alkoxy or thioether group, having from 1 to 6 carbon atoms, preferably from 1 to 4 carbon atoms, such as methoxy, ethoxy, propoxy, isopropoxy, butoxy groups, an alkenyloxy group, preferably an allyloxy group or a phenoxy group,

a cyclohexyl, phenyl or benzyl group,

an acyl group having from 2 to 6 carbon atoms,

a group of formula:

—R₁—OH

—R₁—SH

—R₁—COOR₂

—R₁—CO—R₂

—R₁—CHO

—R₁—CN

—R₁—N(R₂)₂

—R₁—CO—N(R₂)₂

—R₁—SO₃Z

—R₁—SO₂Z

—R₁—X

—R₁—CF₃

in the said formulae R₁ represents a valency bond or a linear or branched, saturated or unsaturated, divalent hydrocarbon group, having from 1 to 6 carbon atoms, such as, for example, methylene, ethylene, propylene, isopropylene, isopropylidene; the R₂ groups, which may be identical or different, represent a hydrogen atom or a linear or branched alkyl group, having from 1 to 6 carbon or phenyl atoms; Z represents a hydrogen atom, an alkali metal preferably sodium or an R₂ group; X symbolizes a halogen atom, preferably a chlorine, bromine or fluorine atom.

The present invention applies quite particularly to halogenoaromatic compounds conforming to the formula (I) in which the R group or groups represent:

a linear or branched alkyl group, having from 1 to 6 carbon atoms, preferably from 1 to 4 carbon atoms, such as methyl, ethyl, propyl, isopropyl, butyl, isobutyl, sec-butyl, tert-butyl,

a linear or branched alkenyl group, having from 2 to 6 carbon atoms, preferably from 2 to 4 carbon atoms, such as vinyl, allyl,

a linear or branched alkoxy group, having from 1 to 6 carbon atoms, preferably from 1 to 4 carbon atoms, such as methoxy, ethoxy, propoxy, isopropoxy, butoxy groups, an alkenyloxy group, preferably an allyloxy group or a phenoxy group,

a group of formula:

—R₁—OH

—R₁—N—(R₂)₂

—R₁—SO₃Z

in the said formulae R₁ represents a valency bond or a linear or branched, saturated or unsaturated divalent hydrocarbon group, having from 1 to 6 carbon atoms, such as, for example, methylene, ethylene, propylene, isopropylene, isopropylidene; the R₂ groups, which may be identical or different, represent a hydrogen atom or a linear or branched alkyl group, having from 1 to 6 carbon or phenyl atoms; Z represents a hydrogen atom or a sodium atom.

In formula (I), n is a number smaller than or equal to 4, preferably equal to 1 or 2.

As examples of compounds conforming to the formula (I), there may be cited in particular p-chlorotoluene, p-bromoanisole, p-bromotrifluorobenzene.

According to the invention, the halogenoaromatic compound of formula (I) reacts with an organometallic compound which conforms to the formula:

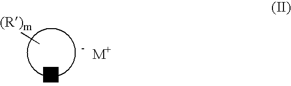

in which:

B symbolizes the remainder of a cycle forming all or part of an aromatic, monocyclic or polycyclic carbocyclic or heterocyclic system,

R′, identical or different, represents substituents on the cycle,

M represents at least one metallic element of group IA of the periodic system,

m represents the number of substituents on the cycle.

For the definition of the elements, reference is made below to the periodic table published in the Bulletin of the Société Chimique de France, no. 1 (1966).

Among the compounds of formula (II), those that are preferred conform to formula (II) in which M represents lithium, sodium, potassium or their mixtures, preferably lithium. Thus, in a simplified manner, “organolithium compound” will be used to describe all compounds conforming to the formula (II).

More precisely, the organolithium compound conforms to the formula (II) in which B represents the remainder of an aromatic carbocyclic or heterocyclic system. Thus, B can acquire the meanings given previously for A. However, B represents more particularly the remainder of a carbocycle such as benzene or napthalene or of a heterocycle such as pyrrole, pyridine, pyrimidine, pyridazine, pyrazine, pyrazole, 1,3-thiazole, 1,3,4-thiadiazole or thiophene, triazole, oxadiazole, pyridazolinone.

The aromatic cycle can also be substituted. The number of substituents m is generally at most 4 per cycle but most often equal to 0 or 1. Reference may be made to the definition of R for examples of substituents.

Preferred substituents are alkyl or alkoxy groups having from 1 to 4 carbon atoms, an amino group, a cyano group, a halogen atom or a trifluoromethyl group.

B preferentially represents the remainder of a benzene ring.

As more particular examples of compounds of formula (II), phenyllithium may be cited in particular.

Compounds conforming to the formula (II) are products which can be obtained by the processes described in the literature in particular by reaction of the alkali metal or an alkyllithium, preferably butyllithium, with an aryl halide [(Modern Synthetic Methods by Manfred Schlosser, p. 233 (1992) Editor Rolf Scheffold)].

The quantity of reagents used is such that the organolithium compound/halogenoaromatic compound molar ratio is advantageously between 0.01 and 3, preferably between 0.75 and 2.

The process of the invention involves a nickel catalyst which can also be in the form of a complex.

The nickel is present with an oxidation number 0. It may be at a greater oxidation number in so far as it is combined with a reducing metal such as for example zinc, manganese and/or magnesium.

Raney nickel can also be used as reducing agent.

In the case where the nickel is used in catalytic quantity, that is to say below the stoichiometric quantity, it must be regenerated during the reaction, combining it likewise with a reducing metal.

It is also to be noted that the presence of an excess of the organolithium compound also permits its reduction to be effected.

As specific examples of derivatives of nickel, there may be cited nickel (II) halides, such as nickel (II) chloride, bromide or iodide: nickel (II) sulphate; nickel (II) carbonate; salts of organic acids comprising from 1 to 18 carbon atoms such as in particular acetate, propionate; nickel (II) complexes such as nickel (II) acetylacetonate, nickel (II) dichloro-bis-(triphenylphosphine), nickel (II) dibromo-bis(bipyridine); nickel (0) complexes such as nickel (0) bis-(cycloocta-1,5-diene), nickel (0) bis-diphenylphosphinoethane.

The nickel can be deposited on a support.

The support is chosen such that it is inert in the conditions of the reaction.

As examples of supports, a mineral or organic support may be used such as in particular carbon, activated carbon, acetylene black, silica, alumina, clays and more particularly montmorillonite or equivalent materials or else a polymeric resin for example a polystyrene.

Generally, the metal is deposited at a rate of 0.5% to 95%, preferably 1% to 5% by weight of the catalyst.

The catalyst can be used in the form of a powder, pellets or else granules.

Complexes of mineral or organic nickel salts can also be used. In these complexes, the ligands or coordination agents are advantageously hydrocarbon derivatives of the elements of column 5.

The said hydrocarbon derivatives of the elements of column 5 derive from valency state III of nitrogen such as nitrogenous amines or heterocycles, of phosphorus such as phosphines, of arsenic such as arsines and of antimony such as stilbines.

They are advantageously chosen from hydrocarbon derivatives of the elements of column VB preferably of a period greater than the 2^{nd}, of nitrogen such as for example bipyridine, bisoxazoline; of phosphorous such as phosphines.

In this last case, this complex is generally realized in situ between the nickel derivative and the phosphine present. But the said complex can also be prepared extemporaneously and introduced into the reaction medium. A supplementary quantity of free phosphine can then be added or not.

Use is advantageously made of aliphatic, cycloaliphatic, arylaliphatic or aromatic phosphines or aliphatic and/or cycloaliphatic and/or arylaliphatic and/or aromatic mixed phosphines.

These phosphines are in particular those which conform to the general formula (III):

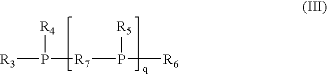

the groups R₃, R₄, R₅, R₆, which may be identical or different, represent:

an alkyl radical having from 1 to 12 carbon atoms,

a cycloalklyl radical having 5 or 6 carbon atoms,

a cycloalklyl radical having 5 or 6 carbon atoms, substituted by one or more than one alkyl radical having from 1 to 4 carbon, alkoxy atoms having 1 or 4 carbon atoms,

a phenylalkyl radical the aliphatic portion of which comprises from 1 to 6 carbon atoms,

a phenyl radical,

a phenyl radical substituted by one or more than one alkyl radical having from 1 to 4 carbon atoms or alkoxy having 1 to 4 carbon atoms.

R₇ represents a valency bond or a divalent, linear or branched, saturated or unsaturated hydrocarbon group, having from 1 to 6 carbon atoms,

q is equal to 0 or 1.

As examples of such phosphines, there may be cited in a non-limiting way: tricyclohexylphosphine, trimethylphosphine, triethylphosphine, tri-n-butyl-phosphine, triisobutylphosphine, tri-tert-butylphosphine, tribenzylphosphine. dicyclohexylphenylphosphine, triphenylphosphine, dimethylphenylphosphine, diethylphenylphosphine, di-tert-butylphenylphosphine.

Nickel (0) tetrakis-(triphenylphosphine) is preferentially used.

As regards the proportions of catalyst, ligand and where necessary reducing metal, it is specified by way of guidance that the quantity of nickel catalyst expressed by the molar ratio between the nickel (expressed as metallic element) and the organolithium compound varies between 5×10⁻⁶ and 0.2, preferably between 5×10⁻⁶ and 0.1, and even more preferentially between 5×10⁻⁶ and 0.05.

The quantity of ligand, preferably a phosphine, used represents from 100 to 500% of the stoichiometric quantity of nickel.

The quantity of reducing metal used represents the stoichiometric quantity necessary to reduce Ni⁺⁺ to Ni₀ up to an excess representing from 100% to 500% of the stoichiometric quantity.

The reaction temperature is advantageously between 70° C. and 150° C., and preferably close to 80° C.

Generally, the reaction is conducted under autogenic pressure of the reagents.

According to a preferred variant of the process of the invention, the process of the invention is carried out under controlled atmosphere of inert gases. An atmosphere of rare gases, preferably argon, can be created, but it is more economical to use nitrogen.

The process according to the invention is carried out in liquid phase.

An inert solvent can be used in the conditions of the reaction of the invention. An aprotic apolar or polar solvent is advantageously used.

As examples of organic solvents suitable for the invention, there may be cited more particularly aliphatic, cycloaliphatic or aromatic hydrocarbons, more particularly petroleum ether, pentane, methylcyclohexane, toluene, xylenes; aliphatic, cycloaliphatic or aromatic ether-oxides, more particularly isopropyl ether, anisole, dioxan, tetrahydrofuran.

The preferred solvents are: toluene, xylenes and methylcyclohexane.

A mixture of organic solvents can also be used.

The concentration of the compounds of formula (I) or (II) used in the solvent can vary within very wide limits. Generally, it varies between 0.1 and 4 mol/l.

From a practical point of view, the process is simple to use.

A preferred embodiment of the invention involves the loading of the organic solvent, the halogenoaromatic compound, the nickel catalyst, the ligand and the progressive addition, for example by pouring, of the organolithium compound in solution or not in an organic solvent.

The reaction mixture is heated and continuously stirred at the reaction temperature.

The mixture is continuously stirred until the reagents are completely consumed, which can be monitored by an analytical method, for example gas-phase chromatography.

Anything which is insoluble (nickel catalyst, zinc salts and zinc) is separated using a solid/liquid separation technique preferably by filtration.

The reaction mass is then treated in standard manner. Water is added, the reaction solvent is evaporated if present and the polyaromatic compound is recovered for example by distillation or crystallization from a suitable solvent, for example an alcohol, in particular methanol, an ester such as isopropyl acetate or water or a mixture of these latter.

The product obtained conforms advantageously to the formula (IV):

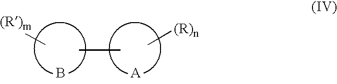

in the said formula R, R′, A, B, n and i have the meaning given above.

The preferred compound conforms to the formula (IV) in which A represents the remainder of a benzene ring.

Embodiments of the example are given below.

In the examples, the abbreviations signify: GPC for gas-phase chromatography and MS for mass spectrometry.

The transformation rate (TR) corresponds to the ratio between the number of transformed substrates and the number of substrate moles involved.

The yield (Y) corresponds to the ratio between the number of moles of product formed and the number of substrate moles (halogenoaromatic compound) involved.

### COMPARATIVE EXAMPLE 1

Synthesis of 4-methylbiphenyl

0.266 g (2.1×10⁻³ mol, 1 eq.) of p-chlorotoluene in 30 ml of anhydrous benzene are loaded into a 100-ml reactor fitted with a magnetic stirrer, equipped with a condenser, a thermocontact, and kept under nitrogen atmosphere.

The mixture is raised to 65° C. under magnetic stirring (600 rpm).

1.75 ml (3.15×10⁻³ mol, 1.5 eq) of a 1.8 M phenyllithium commercial solution (in the mixture cyclohexane 70%/ether 30%) are added in 20 min. at 65° C. and under nitrogen.

The reaction medium is kept at 65° C., under magnetic stirring and under nitrogen for 48 h.

The mixture is then cooled to 25° C. 50 ml of water and 25 ml of ether are added to the reaction medium, and the latter is then neutralized at pH 6-7 with a 0.1 N hydrochloric acid solution.

The organic phase is separated, the aqueous phase is extracted with three times 75 ml of ether.

The collected organic phases are washed with a saturated solution of sodium chloride, dried over anhydrous magnesium sulphate, filtered and evaporated.

The compounds are identified in GPC/MS and in GPC by co-injection with a standard sample of 4-methylbiphenyl.

The residue is determined in gas-phase chromatography with naphthalene as standard.

4-methylbiphenyl is obtained with a yield of 24%. 3-methylbiphenyl is obtained with a yield of 3%.

### EXAMPLE 2

Synthesis of 4-methylbiphenyl

55.9 mg (8.55×10⁻⁵ mol, 0.04 eq.) of nickel (II) dichloro-bis-(triphenyl-phosphine) in 30 ml of anhydrous benzene are loaded into a 100-ml reactor fitted with a magnetic stirrer, equipped with a condenser, a thermocontact, and kept under nitrogen atmosphere.

0.27 g (2.14×10⁻³ mol, 1 eq.) of p-chlorotoluene are added at 25° C. and under nitrogen.

The mixture is raised to 65° C. under magnetic stirring (600 rpm).

1.88 ml (3.21×10⁻³ mol, 1.5 eq) of a 1.7 M phenyllithium commercial solution (in the mixture cyclohexane 70%/ether 30%) are added in 20 min. at 65° C. and under nitrogen.

The reaction medium is left at 65° C., under magnetic stirring and under nitrogen for 3 h.

The mixture is then cooled to 25° C. 50 ml of water and 25 ml of ether are added to the reaction medium, and the latter is then neutralized at pH 6-7 with a 0.1 N hydrochloric acid solution.

The organic phase is separated, the aqueous phase is extracted with three times 75 ml of ether.

The collected organic phases are washed with a saturated solution of sodium chloride, dried over anhydrous magnesium sulphate, filtered and evaporated.

The compound is identified in GPC/MS and in GPC by co-injection with a standard sample of 4-methylbiphenyl.

The residue is determined in gas-phase chromatography with naphthalene as standard.

4-methylbiphenyl is obtained with a yield of 55%.

### EXAMPLE 3

Synthesis of 4-methylbiphenyl

68.6 mg (1.29.⁻⁴ mol, 0.04 eq.) of nickel (II) dibromo-bis-(bipyridine) in 25 ml of anhydrous benzene are loaded into a 100-ml reactor fitted with a magnetic stirrer, equipped with a condenser, a thermocontact, and kept under nitrogen atmosphere.

0.408 g (3.23×10⁻³ mol, 1 eq.) of p-chlorotoluene are added at 25° C. and under nitrogen.

The mixture is raised to 65° C. under magnetic stirring (600 rpm).

2.7 ml (4.85×10⁻³ mol, 1.5 eq) of a 1.8 M phenyllithium commercial solution (in the mixture cyclohexane 70%/ether 30%) are added in 20 min. at 65° C. and under nitrogen.

The reaction medium is left at 65° C., under magnetic stirring and under nitrogen for 3 h.

The mixture is then cooled to 25° C. 50 ml of water and 25 ml of ether are added to the reaction medium, and the latter is then neutralized at pH 6-7 with a 0.1 N hydrochloric acid solution.

The organic phase is separated, the aqueous phase is extracted with three times 75 ml of ether.

The collected organic phases are washed with a saturated solution of sodium chloride, dried over anhydrous magnesium sulphate, filtered and evaporated.

The compound is identified in GPC/MS and in GPC by co-injection with a standard sample of 4-methylbiphenyl.

The residue is determined in gas-phase chromatography with naphthalene as standard.

4-methylbiphenyl is obtained with a yield of 41%.

### EXAMPLE 4

Synthesis of 4-methylbiphenyl

56.7 mg (7.63×10⁻⁵ mol, 0.04 eq.) of nickel (II) dibromo-bis-(triphenylphosphine) in 25 ml of anhydrous benzene are loaded into a 100-ml reactor fitted with a magnetic stirrer, equipped with a condenser, a thermocontact, and kept under nitrogen atmosphere.

0.245 g (1.9×10⁻³ mol, 1 eq.) of p-chlorotoluene are added under nitrogen and at 25° C.

The mixture is raised to 65° C. under magnetic stirring (500 rpm).

1.6 ml (2.86×10⁻³ mol, 1.5 eq) of a 1.8 M phenyllithium solution (in the mixture cyclohexane 70%/ether 30%) are added in 20 min. at 65° C. and under nitrogen.

The reaction medium is left at 65° C., under magnetic stirring and under nitrogen for 3 h.

The mixture is then cooled to 25° C. 75 ml of water and 50 ml of ether are added to the reaction medium, and the latter is then neutralized at pH 6-7 with a 0.1 N hydrochloric acid solution.

The organic phase is separated, the aqueous phase is extracted with three times 75 ml of ether.

The collected organic phases are washed with a saturated solution of sodium chloride, dried over anhydrous magnesium sulphate, filtered and evaporated.

The compound is identified in GPC/MS and in GPC by co-injection with a standard sample of 4-methylbiphenyl.

The residue is determined in gas-phase chromatography with naphthalene as standard.

4-methylbiphenyl is obtained with a yield of 73%.

### EXAMPLE 5

Synthesis of 4-methylbiphenyl

16.5 mg (2.22×10⁻⁵ mol, 0.008 eq.) of nickel (II) dibromo-bis-(triphenylphosphine) in 25 ml of anhydrous benzene are loaded into a 100-ml reactor fitted with a magnetic stirrer, equipped with a condenser, a thermocontact, and kept under nitrogen atmosphere.

0.347 g (2.75×10⁻³ mol, 1 eq.) of p-chlorotoluene are added at 25° C. and under nitrogen.

The mixture is raised to 65° C. under magnetic stirring (600 rpm).

2.31 ml (4.15×10⁻³ mol, 1.5 eq) of a 1.8 M phenyllithium solution (in the mixture cyclohexane 70%/ether 30%) are added in 20 min. at 65° C. and under nitrogen.

The reaction medium is left at 65° C., under magnetic stirring and under nitrogen for 3 h.

The mixture is then cooled to 25° C., 50 ml of water and 50 ml of ether are added to the reaction medium, and the latter is then neutralized at pH 6-7 with a 0.1 N hydrochloric acid solution.

The organic phase is separated, the aqueous phase is extracted with three times 50 ml of ether.

The collected organic phases are washed with a saturated solution of sodium chloride, dried over anhydrous magnesium sulphate, filtered and evaporated.

The compound is identified in GPC/MS and in GPC by co-injection with a standard sample of 4-methylbiphenyl.

An aliquot of the residue is determined in gas-phase chromatography with naphthalene as standard.

4-methylbiphenyl is obtained with a yield of 75%.

### EXAMPLE 6

Synthesis of 4-methoxybiphenyl

197 mg (2.65×10⁻⁴ mol, 0.04 eq.) of nickel (II) dibromo-bis-(triphenylphosphine) in 40 ml of anhydrous benzene are loaded into a 250-ml reactor fitted with a magnetic stirrer, equipped with a condenser, a thermocontact, and kept under nitrogen atmosphere.

0.83 g (6.63×10⁻³ mol, 1 eq.) of p-bromoanisole are added at 25° C. and under nitrogen.

The mixture is raised to 65° C. under magnetic stirring (600 rpm).

6.3 ml (1.13×10⁻² mol, 1.7 eq) of a 1.8 M phenyllithium commercial solution (in the mixture cyclohexane 70%/ether 30%) are added in 20 min. at 65° C. and under nitrogen.

The reaction medium is left at 65° C., under magnetic stirring and under nitrogen for 2 h.

The mixture is then cooled to 25° C., 100 ml of water are added to the reaction medium, and the latter is then neutralized at pH 6-7 with a 0.1 N hydrochloric acid solution.

The organic phase is separated, the aqueous phase is extracted with three times 100 ml of ether.

The collected organic phases are washed with a saturated solution of sodium chloride, dried over anhydrous magnesium sulphate, filtered and evaporated.

The residue is purified by chromatography over a silica column (eluant: hexane).

4-methoxyylbiphenyl is obtained with an isolated yield of 56%.

### EXAMPLE 7

Synthesis of 4-trifluoromethylbiphenyl

198.6 mg (2.67×10⁻⁴ mol, 0.04 eq.) of nickel (II) dibromo-bis-(triphenylphosphine) in 40 ml of anhydrous benzene are loaded into a 250-ml reactor fitted with a magnetic stirrer, equipped with a condenser, a thermocontact, and kept under nitrogen atmosphere.

0.94 ml (6.69×10³ mol, 1 eq.) of p-bromotrifluoromethylbenzene are added at 25° C. under nitrogen.

The mixture is raised to 65° C. under magnetic stirring (600 rpm).

6.3 ml (1.14×10⁻² mol, 1.7 eq) of a 1.8 M phenyllithium commercial solution (in the mixture cyclohexane 70%/ether 30%) are added in 30 min. under nitrogen and at 65° C.

The reaction medium is left at 65° C., under magnetic stirring and under nitrogen for 2 h.

The mixture is then cooled to 25° C. 150 ml of water are added to the reaction medium, and the latter is then neutralized at pH 6-7 with a 0.1 N hydrochloric acid solution. The organic phase is separated, the aqueous phase is extracted with three times 100 ml of ether.

The collected organic phases are washed with a saturated solution of sodium chloride, dried over anhydrous magnesium sulphate, filtered and evaporated.

The compound is identified in GPC/MS and in GPC by co-injection with a standard sample of 4-trifluoromethylbiphenyl.

4-trifluoromethylbiphenyl is obtained with a yield of 90% by GPC determination (TF=95% by NMR¹⁹F determination).

A chromatography over a silica column (eluant: hexane) allows the compound to be obtained with an isolated yield of 7%.

1- Preparation process for a polycyclic aromatic compound comprising at least a two linked aromatic cycles characterized in that it involves reacting an aromatic compound bearing a parting group and an alkaline organometallic compound, in the presence of an effective quantity of a nickel catalyst, the said element being optionally complexed with at least one coordination agent or ligand.

2- Process according to claim 1 characterized in that the halogeno-aromatic compound conforms to the general formula (1): 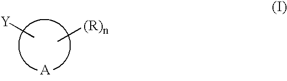
in which:
A symbolizes the remainder of a cycle forming all or part of an aromatic, monocyclic or polycyclic carbocyclic or heterocyclic system,
R, identical or different, represent substituents on the cycle,
Y represents a parting group, preferably a halogen atom or a sulphonic ester group of formula —OSO₂—R, in which R is a hydrocarbon group,
n represents the number of substituents on the cycle.

3- Process according to claim 2 characterized in that the halogeno-aromatic compound conforms to the formula (I) in which Y is a bromine or chlorine atom or a sulphonic ester of formula —OSO₂—R, in which R is a linear or branched alkyl group having from 1 to 4 carbon atoms, preferably a methyl or ethyl group, a phenyl or tolyl group or a trifluoromethyl group.

4- Process according to one of claims 2 and 3 characterized in that the halogenoaromatic compound conforms to the formula (I) in which A is the remainder of a cyclic compound, preferably having at least 4 atoms in the cycle, preferably 5 or 6, optionally substituted, and representing at least one of the following cycles:
a monocyclic or polycyclic aromatic carbocycle,
a monocyclic or polycyclic aromatic heterocycle comprising at least one of the heteroatoms O, N and S.

5- Process according to one of claims 2 to 4 characterized in that the halogenoaromatic compound conforms to the formula (I) in which the optionally substituted remainder A represents an aromatic carbocycle, an aromatic bicycle comprising two aromatic carbocycles, a partially aromatic bicycle comprising two carbocycles in which one of the two is aromatic, an aromatic heterocycle, an aromatic bicycle comprising an aromatic carbocycle and an aromatic heterocycle, a partially aromatic bicycle comprising an aromatic carbocycle and a heterocycle, an aromatic bicycle comprising two aromatic heterocycles, a partially aromatic bicycle comprising a carbocycle and an aromatic heterocycle, a tricycle comprising at least one carbocycle or an aromatic heterocycle.

6- Process according to one of claims 2 to 4 characterized in that the halogenoaromatic compound conforms to the formula (I) in which A represents a benzene or naphthalene ring.

7- Process according to one of claims 2 to 6 characterized in that the halogenoaromatic compound of formula (I) bears one or more than one substituent such as:
a linear or branched alkyl group, having from 1 to 6 carbon atoms, preferably from 1 to 4 carbon atoms, such as methyl, ethyl, propyl, isopropyl, butyl, isobutyl, sec-butyl, tert-butyl,
a linear or branched alkenyl or alkynyl group, having from 2 to 6 carbon atoms, preferably from 2 to 4 carbon atoms, such as vinyl, allyl,
a linear or branched alkoxy or thioether group, having from 1 to 6 carbon atoms, preferably from 1 to 4 carbon atoms, such as methoxy, ethoxy, propoxy, isopropoxy, butoxy groups, an alkenyloxy group, preferably an allyloxy group or a phenoxy group,
a cyclohexyl, phenyl or benzyl group,
an acyl group having from 2 to 6 carbon atoms,
a group of formula:
—R₁—OH
—R₁—SH
—R₁—COOR₂
—R₁—CO—R₂
—R₁—CHO
—R₁—CN
—R₁—N(R₂)₂
—R₁—CO—N(R₂)₂
—R₁—SO₃Z
—R₁—SO₂Z
—R₁—X
—R₁—CF₃
in the said formulae R₁ represents a valency bond or a linear or branched, saturated or unsaturated, divalent hydrocarbon group, having from 1 to 6 carbon atoms, such as, for example, methylene, ethylene, propylene, isopropylene, isopropylidene; the R₂ groups, which may be identical or different, represent a hydrogen atom or a linear or branched alkyl group, having from 1 to 6 carbon or phenyl atoms; Z represents a hydrogen atom, an alkali metal preferably sodium or an R₂ group; X symbolizes a halogen atom, preferably a chlorine, bromine or fluorine atom.

8- Process according to one of claims 2 to 7 characterized in that the halogenoaromatic compound conforms to the formula (I) in which n is a number smaller than or equal to 4, preferably equal to 1 or 2.

9- Process according to one of claims 2 to 8 characterized in that the halogenoaromatic compound of formula (I) is chosen from: p-chlorotoluene, p-bromoanisole, p-bromotrifluorobenzene.

10- Process according to claim 1 characterized in that the alkaline organometallic compound conforms to the formula (II): 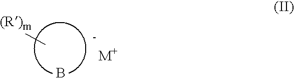
in which:
B symbolizes the remainder of a cycle forming all or part of an aromatic, monocyclic or polycyclic carbocyclic or heterocyclic system,
R′, identical or different, represent substituents on the cycle,
M represents at least one metallic element of group LA of the periodic table,
m represents the number of substituents on the cycle.

11- Process according to claim 10 characterized in that the alkaline organometallic compound conforms to the formula (II) in which M represents lithium.

12- Process according to claim 10 characterized in that the alkaline organometallic compound conforms to the formula (II) in which B represents the remainder of a carbocycle such as benzene or napthalene or of a heterocycle such as pyrrole, pyridine, pyrimidine, pyridazine, pyrazine, pyrazole, 1,3-thiazole, 1,3,4-thiadiazole or thiophene, triazole, oxadiazole, pyridazolinone.

13- Process according to one of claims 10 to 12 characterized in that the alkaline organometallic compound presents an aromatic cycle bearing at least one substituent chosen from alkyl or alkoxy groups having from 1 to 4 carbon atoms, an amino group, a cyano group, a halogen atom or a trifluoromethyl group.

14- Process according to claim 10 characterized in that the alkaline organometallic compound conforms to the formula (II) in which m is a number smaller than or equal to 4, preferably equal to 0 or 1.

15- Process according to one of claims 10 to 14 characterized in that the alkaline organometallic compound is phenyllithium.

16- Process according to one of claims 1 to 15 characterized in that the quantity of reagents used is such that the alkaline organometallic compound/halogenoaromatic compound molar ratio is between 0.01 and 3, preferably between 0.75 and 2.

17- Process according to one of claims 1 to 16 characterized in that the nickel catalyst comprises nickel with an oxidation number of 0 or nickel with a greater oxidation number combined with a reducing metal, preferably zinc, manganese and/or magnesium or else Raney nickel.

18- Process according to one of claims 1 to 17 characterized in that the nickel catalyst is chosen from nickel (II) halides, such as nickel (II) chloride, bromide or iodide: nickel (II) sulphate; nickel (II) carbonate; salts of organic acids comprising from 1 to 18 carbon atoms such as in particular acetate, propionate; nickel (II) complexes such as nickel (II) acetylacetonate, nickel (II) dichloro-bis-(triphenylphosphine), nickel (II) dibromo-bis(bipyridine); nickel (0) complexes such as nickel (0) bis-(cycloocta-1,5-diene), nickel (0) bis-diphenylphosphinoethane.

19- Process according to claim 18 characterized in that the nickel catalyst is nickel (II) chloride combined with a reducing agent, preferably zinc.

20- Process according to one of claims 1 to 19 characterized in that the nickel is in the form of complexes in which the ligand is a hydrocarbon derivative of the elements of column V derived from valency state III of nitrogen, phosphorous, arsenic or antimony.

21- Process according to claim 20 characterized in that the ligand is an aliphatic, cycloaliphatic, arylaliphatic or aromatic phosphine or an aliphatic and/or cycloaliphatic and/or arylaliphatic and/or aromatic mixed phosphine.

22- Process according to claim 21 characterized in that he phosphine used is chosen from tricyclohexylphosphine, trimethylphosphine, triethyl-phosphine, tri-n-butylphosphine, triisobutylphosphine, tri-tert-butylphosphine, tribenzylphosphine, dicyclohexylphenylphosphine, triphenylphosphine, dimethyl-phenylphosphine, diethylphenylphosphine, di-tert-butylphenylphosphine.

23- Process according to one of claims 1 to 22 characterized in that the quantity of nickel catalyst expressed by the molar ratio between the nickel and the alkaline organometallic compound varies between 5×10⁻⁶ and 0.2, preferably between 5×10⁻⁶ and 0.1, and even more preferentially between 5×10⁻⁶ and 0.05.

24- Process according to one of claims 1 to 23 characterized in that the quantity of ligand, preferably a phosphine, used represents from 100 to 500% of the stoichiometric quantity of nickel.

25- Process according to one of claims 1 to 24 characterized in that the quantity of reducing metal used represents the stoichiometric quantity necessary to reduce Ni⁺⁺ to Ni₀ up to an excess representing from 100% to 500% of the stoichiometric quantity.

26- Process according to one of claims 1 to 25 characterized in that the reaction temperature is between 70° C. and 150° C., and preferably close to 80° C.

27- Process according to one of claims 1 to 26 characterized in that the reaction is conducted in an aprotic apolar or polar solvent preferably chosen from aliphatic, cycloaliphatic or aromatic hydrocarbons, more preferentially petroleum ether, pentane, methylcyclohexane, toluene, xylenes; aliphatic, cycloaliphatic or aromatic ether-oxides, more preferentially isopropyl ether, anisole, dioxan, tetrahydrofuran.

28- Process according to one of claims 1 to 27 characterized in that the product obtained conforms advantageously to the formula (IV): 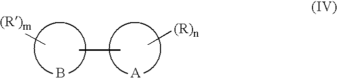
in the said formula R, R′, A, B, n and m have the meaning given above in one of claims 2 to 8 and 10 to 14.

29- Process according to claim 28 characterized in that the compound of formula (IV) is 4-methylbiphenyl, 4-methoxybiphenyl, 4-trifluoro-methylbiphenyl.
